(19) United States
(12) Patent Application Publication
(10) Pub. No.: US 2024/0314440 A1
(43) Pub. Date: Sep. 19, 2024

(54) Method for Controlling a Camera Robot

(71) Applicant: ROBIDIA GMBH, Köln (DE)

(72) Inventor: Masih Jakubi, Köln (DE)

(73) Assignee: ROBIDIA GMBH, Köln (DE)

(21) Appl. No.: 18/279,663

(22) PCT Filed: Mar. 3, 2021
(86) PCT No.: PCT/EP2021/055353

§ 371 Date: Aug. 31, 2023

(51) Int. Cl. H04N 23/695; F16M 11/12; F16M 11/42; H04N 23/57; H04N 23/61; H04N 23/62; H04N 23/66

(52) U.S. Cl. CPC H04N 23/695; F16M 11/12; H04N 23/61; F16M 11/42; F16M 2200/06; H04N 23/57; H04N 23/62; H04N 23/66

(57) ABSTRACT

The invention relates to a method for controlling a camera robot for shooting a video sequence, the camera robot including
a chassis that can be moved on a surface;
a camera for shooting the video sequence;
a holding device for connecting the camera to the chassis as well as for orienting the camera relative to the chassis; and
a control unit configured to control the chassis, the holding device and/or the camera;
the method including the following steps
   determining a characteristic shooting scene; and
   determining control parameters of the control unit depending on the determined characteristic shooting scene.

### CROSS REFERENCE TO RELATED APPLICATION

This application is the United States national phase of International Patent Application No. PCT/EP2021/055353 filed Mar. 3, 2021, the disclosure of which is hereby incorporated by reference in its entirety.

### BACKGROUND OF THE INVENTION

### Field of the Invention

The present invention relates to a method for controlling a camera robot for shooting a video sequence as well as a corresponding camera robot.

### Description of Related Art

Usually, a cinematographer is used for shooting video sequences who, depending on the given shooting scene, guides the camera such that optimal shooting effects are achieved for the corresponding shooting scene.

For example, in a romantic shooting scene characterized by a candlelight dinner, it may be provided that the camera is guided along a circular path, with the camera always being directed toward the protagonists involved in the scene. In this shooting scene, the cinematographer has to guide the translational movement of the camera along the circular path as precisely as possible while tracking the pan angle of the camera in order to realize a total 360° travel of the camera for the desired shooting. This requires an experienced cinematographer, who is also required to have sufficient concentration throughout the entire shooting day. In the 360° camera travel described above, it may also be particularly important to maintain a constant distance between the camera and the subject in order to achieve the desired effect. Deviations from the ideal route may lead to undesired effects.

Similarly, other shooting scenes may require the camera to be guided along an arc-shaped path, wherein the arc shape corresponds to an ellipse segment, for example.

In this case, precise guidance of the camera and, if necessary, simultaneous adjustment of the pan angle of the camera are also indispensable to achieve the desired effect.

Moreover, it is important to adjust the speed or acceleration of the camera to suit the respective shooting scene. For example, in the romantic shooting scene described in detail, it may be necessary to move the camera at a relatively low speed to achieve the desired scene effect, while in an action scene it may be necessary to set a higher camera travel speed.

The shooting scenes exemplarily descried above make clear that the shooting of video sequences according to the methods used so far requires the use of an experienced cinematographer and that the correct shooting of a video sequence is a demanding task. In this respect, it is hardly surprising that during shooting a scene often has to be shot several times until the desired result is achieved.

### SUMMARY OF THE INVENTION

Based on the above problem, it is the object of the present invention to provide a method that allows efficient shooting of video sequences.

The above-mentioned object is achieved by proposing a method for controlling a camera robot for shooting a video sequence, the camera robot comprising:
a chassis that can be moved on a surface;
a camera for shooting the video sequence;
a holding device for connecting the camera to the chassis as well as for orienting the camera relative to the chassis; and
a control unit configured to control the chassis, the holding device and/or the camera;
   the method comprising the following steps:
determining a characteristic shooting scene; and
determining control parameters of the control unit depending on the determined characteristic shooting scene.

The method according to the invention offers the advantage that video sequences can be shot in a particularly efficient manner. The use of a camera robot allows the camera travel to be automated. Moreover, the use of an automatically controlled chassis allows a higher precision to be achieved in camera travel than is usually possible with manual shooting with the aid of a cinematographer. Overall, fewer shooting attempts are needed to achieve the desired result of shooting a video sequence. Thus, the required shooting time is significantly reduced. At the same time, the production costs are also reduced. By determining the characteristic shooting scene and determining and adjusting control parameters of the control unit depending on the determined characteristic shooting scene, it is achieved that empirical values from past shootings can be profited from and that the control parameters that have proven to be particularly suitable for a certain shooting scene in previous shootings can be used again in future shootings. This allows for a high degree of automation when shooting video sequences as well as an increased efficiency in the shooting process.

In the context of the present invention, two different approaches can be applied. On the one hand, it may be provided according to the method of the invention that the desired shooting scene is manually input by a user and then the control parameters corresponding to the current shooting scene are loaded from a database. Alternatively, in the context of the present invention, it may be provided that the shooting scene is determined in an automated manner, particularly by evaluating image shots or video shots of the current shooting scene.

The holding device used in the present invention can comprise a fastening means for fastening to the chassis, for example. This allows the camera to be stably fastened to the chassis. In addition, the holding device can comprise a linear motor-like device configured to move the camera along a vertical axis. For example, the holding device can comprise a lifting column. Furthermore, the holding device can comprise one or more pan motors configured to adjust one or more pan angles of the camera. This allows the camera to be oriented relative to the chassis, both in terms of its height and its pan angle.

The control unit can be configured to only control the chassis. Alternatively, the control unit can be configured to control both the chassis and the holding device. According to the present invention, it may also be provided that the control unit is configured to control the chassis, the holding device, and the camera. After determining the control parameters, the latter can be transmitted to the chassis, the holding device and/or the camera in order to control the desired component according to the determined shooting scene.

According to an embodiment of method of the invention, it may be provided that the characteristic shooting scene is determined by evaluating a user input. Here, it may be provided that the user manually specifies the desired shooting scene via a user interface, for example via a graphical user interface (GUI). In this embodiment, the use of artificial intelligence for determining the characteristic shooting scene is not required. Rather, it is possible, for example, to determine which control parameters are to be regarded as optimal for the selected shooting scene by using a database that stores the assignment between shooting scenes and corresponding control parameters. In particular, the control parameters can not only be static control parameters, but also control parameters that change over time. For example, the control parameters can store information on the required movement of the camera robot and the tilt angle of the camera. Thus, it can be coded in a control parameter dataset along which route the camera robot should move and how one or more pan angles of the camera should be set in terms of time. The advantage of the embodiment of the present invention, in which the characteristic shooting scene is determined by evaluating a user input, is to be seen in the fact that no previously trained system has to be used which first learns a reliable recognition of the shooting scene by machine learning. This makes an implementation of the method particularly simple and the technical requirements are greatly reduced. Furthermore, the user is given control over the selection of the shooting scene. This also reduces any residual risk of incorrect recognition of the shooting scene.

According to a further embodiment of method of the invention, it may be provided that the characteristic shooting scene is determined by a method based on machine learning. Unlike the previous embodiment, the shooting scene in this embodiment is recognized by using artificial intelligence. In this way, information or empirical values gathered during previous shootings are used to determine the optimal control parameters for future shootings in an automated manner.

According to an advantageous embodiment of the method of the invention, it may be provided that, when determining the characteristic shooting scene, a system previously trained with training data is used which was trained during a training process with image data or video sequences and corresponding labels which identify the affiliation of the image data or video sequences to the shooting scenes. In particular, the training data may have been shot in previous shootings. For example, in the previous shootings where a cinematographer was used to manually set the “control parameters” (especially the position and orientation of the camera), sensors may have been used to detect each of the parameters set. In particular, acceleration sensors may be provided on the camera for shooting the training data, which detect the position of the camera in a horizontal plane, the vertical height of the position of the camera, and the orientation or pan angle of the camera. In this way, the cinematographer's camera work can be evaluated in corresponding shooting scenes, so that a system trained by the manually set parameters can be provided for future shootings. The system can be trained with static image data or video sequences as well as control parameters or entire control parameter datasets associated with the image data or video data. Static image data can already provide characteristic information about which shooting scene is currently present. The image data can either be used directly to train the system or indirectly by extracting individual image parameters. When the system is directly trained with image data, information about which shooting scene is involved can be obtained based on the brightness or illumination of the image. For example, in a cooking scene or an interview, the image is typically more brightly lit than in a horror shooting scene, for example. The colour representation also contains relevant information that is typical for the atmosphere in a specific shooting scene. The image composition can also contain important information for recognizing the shooting scene, wherein certain objects can be recognized that are characteristic for a specific shooting scene. If, for example, there is knife or a weapon in an image, it can be inferred that this is possibly a horror scene. If there are candles in an image, this may suggest that this is a romantic shooting scene. In addition to the objects in an image, the orientation of the images can also contain relevant information that is typical for a specific shooting scene. For example, the posture of a knife in a horror scene may be different from the posture of the knife in a cooking show. Furthermore, the posture of the protagonists and their facial expressions can contain relevant information that indicate the atmosphere in the scene. For example, the facial expression of a cook who is holding a knife in his hand differs from the facial expression of an actor in the role of a murderer. In addition, the scene dynamics can provide further information about the present shooting scene. Here, video sequences can be evaluated from which it can be determined whether the scene is dynamic or rather static. For example, the dynamics of the recorded video sequence can be used to determine whether it is a romantic scene or an action scene.

According to an embodiment of the method of the invention, it may be provided that the characteristic shooting scene comprises a total of two shooting scenes. For example, the shooting scenes can be an action scene and a romantic scene. It is obvious to the person skilled in the art that particularly high recognition rates can be achieved if only a few shooting scenes have to be distinguished. Therefore, it is advantageously achieved that even little training data can lead to sufficiently high recognition rates.

According to a further embodiment of the method of the invention, it may be provided that the characteristic shooting scene comprises one of the following shooting scenes:
action,
horror,
romance,
dance,
presentation,
interview, or
panel discussion.

For example, an action scene can be recognized by detecting the shooting dynamics typical of an action scene. In other words, information about how fast an object moves from a starting point to a target point can provide information about the dynamics of the present shooting scene. Here it is therefore necessary that video sequences are evaluated. Alternatively, the information about how many frames are needed until an object is moved from a starting position to a target position can provide relevant information about the dynamics of the present shooting scene. Furthermore, information on the protagonists' facial expressions, gestures and/or posture can provide characteristic information that indicates the presence of an action scene. Specific objects, such as a weapon, can also provide clues that the scene is an action scene. Events such as an explosion can also suggest that the scene is an action scene. Furthermore, horror scenes, for example, can be recognized when sudden changes are detected. For example, a horror scene can be inferred if an attacker suddenly appears in the image. The evaluation of video sequences is particularly helpful for this purpose. A romantic scene can be inferred, for example, if the protagonists' gestures and facial expressions are typical of a romantic scene. A smile, the proximity of the protagonists to each other and/or their posture can provide characteristic information. Furthermore, an algorithm for skeleton recognition can be used to evaluate the posture of the protagonists. Also, the recognition of an embrace may suggest a romantic scene. Furthermore, characteristic features, such as the direction of the protagonists' gaze or also the lighting, especially through warm colours or also a low dynamic, can indicate a romantic scene.

For example, if a romantic scene is recognized, the control parameters that are considered appropriate for such a romantic scene can be loaded. In these control parameters, for example, a zoom travel, travelling along a semicircle, or a 360° travel can be codified. Here, either fixed control parameters can be loaded or alternatively the determined control parameters can be adapted to the given situation. In this way, for example, objects that are considered disturbing during the determined camera travel can be bypassed. For this purpose, for example, the control parameters that relate to the position of the camera in the horizontal plane or the vertical position of the camera can be adapted accordingly.

Dance scenes can be detected, for example, if a dynamic typical of dance scenes is recognized and also a corresponding posture of the protagonists that are characteristic of dances. Dance scenes can also be more finely subdivided into specific dance styles (e.g. flamenco, salsa or hip-hop). Provided that a dance scene has been determined or set in advance by the user, specific control parameters can be determined that have proven effective for shooting dance scenes. For example, in this case 360° travels can be performed or the dancers can be tracked. The control parameters may differ for different dances, for example if a very slow or a very dynamic dance is to be shot. Another example is the presentation scene, where, for example, one or more persons are recognized whose gazes point in the same direction. If a presentation scene has been recognized or input by the user, for example, a camera travel can be performed along an arc-shaped trajectory.

On the other hand, it can be detected that the preset scene is an interview scene, for example, if several people are recognized who are positioned at a distance from each other, who are typical for an interview and whose posture and orientation towards each other are characteristic for an interview. In addition, the lighting may include characteristic information that suggest the presence of an interview scene.

As explained above, automatic detection is to be understood as an option in the context of the present invention. In the same way, it can also be provided that the user selects the shooting scene manually and that the control parameters associated with the selected shooting scene are determined according to the method according to the invention.

According to a further embodiment of the method according to the invention, it may be provided that the control parameters are determined depending on the determined characteristic shooting scene by a method based on machine learning, wherein in particular a system previously trained with training data is used which was trained during a training process with image data or video sequences as well as shooting scene information and control parameters. As already mentioned above, sensors can be used to record the training data, allowing the control parameters to be recorded. Thus, several romantic scenes can be shot manually, wherein, for example, the captured image data are stored and classified as romance image shots. Classification can be done manually by an experienced user or by a cinematographer who can reliably distinguish the shooting scenes. The control parameters belonging to the shooting scene are also stored. Through this stored training data, the method based on machine learning can learn which control parameters are suitable for a specific shooting scene.

Furthermore, several acceleration sensors can be arranged on the camera, which detect the exact position or orientation of the camera. For example, in a film studio, several hundred shooting scenes of a first scene type can be shot with the corresponding control parameters, which were set manually by a cinematographer. Then several hundred shooting scenes of another scene type can be shot with the corresponding control parameters. In doing so, the shooting scene information can be determined manually. So the knowledge of the corresponding shooting scene is used. The trained system thus teaches which control parameters can be considered suitable for the respective shooting scene by feeding in the control parameters and the shooting scene information associated with the control parameters. The shooting scene information can be marked as “action”, “horror”, “romance” etc. according to the respective shooting scenes.

According to a further embodiment of the method according to the invention, it may be provided that determining the control parameters includes determining chassis control parameters which are used to control the movement of the chassis on the surface along a determined route. In particular, the chassis control parameters may include information comprising the position of the camera on a horizontal plane. This allows all the parameters necessary for a particular camera travel to be included in the chassis control parameters. According to the above-described example, the chassis control parameters may include the positions of the camera that need to be travelled in order to realize a 360° travel. Several hundred or even several thousand positions can be stored, which enable to travel along the circular shape in a particularly precise manner.

According to a further exemplary embodiment of the method according to the invention, it may be provided that chassis control parameters are determined by a method based on machine learning, wherein a system previously trained with training data is used which was trained during a training process with image data or video sequences as well as chassis control parameters. In this way, during the training process, the chassis control parameters that are typical for a particular shooting scene can be read out. Thus, the experience gained by a cinematographer during previous shootings can be used to enable camera travels in automated form in future shootings. For example, if a 360° travel was performed during a romantic scene, the trained system can infer that a 360° travel is appropriate during a future detection of a romantic scene and subsequently perform such a camera travel in a fully automated manner.

According to an embodiment of the method according to the invention, it may be provided that objects located in the environment of the camera robot are detected, and that chassis control parameters are determined depending on the detected objects and/or their position. In this way, the initial determined camera travel can be adjusted depending on the detected objects. For example, if it has been determined that a 360° travel is to be performed, but there are obstacles on the determined route, it can be taken into account before the travel is carried out that the detected objects will obstruct the route that was initially found to be optimal. For example, a 360° travel can be performed, which takes place along a circular path that has a radius that differs from the radius previously considered ideal. For example, if it was initially determined that a 360° travel along a circular path corresponding to a circle with a radius of 1 m was considered optimal, but obstacles are detected on this circular path, a travel along a circular arc corresponding to a circle with a radius of 1.20 m can be performed, for example. In addition, the camera settings can optionally be adjusted so that the increased distance to the subject is compensated for by appropriate zoom parameters.

According to a further embodiment of the method according to the invention, it may be provided that the objects located in the environment of the camera robot are detected by using a LIDAR sensor. By using a LIDAR sensor, it can be achieved in an advantageous manner that any obstacles in the room can be reliably detected.

According to a further embodiment of the method according to the invention, it may be provided that the holding device is configured to adjust the position of the camera along a vertical axis and/or a pan angle of the camera, and that determining the control parameters includes determining holding device control parameters used to control the position of the camera along a vertical axis and/or the pan angle of the camera. This not only allows the route of the robot to be determined automatically, but also the height position of the camera and its pan angle. For this purpose, the holding device can in particular have a linear motor-like device for adjusting the height position of the camera, such as a lifting column. In addition, the holding device can in particular have one or two pan motors to adjust the pan angle(s) of the camera.

According to a further embodiment of the method according to the invention, it may be provided that holding device control parameters are determined by a method based on machine learning, wherein a system previously trained with training data is used which was trained during a training process with image data or video sequences as well as holding device control parameters. In this way, during the training process, the camera work can be learned depending on the image data or video sequences and the associated shooting scenes. The basis for the learning process is provided by the cinematographer who manually guided the camera during earlier shootings. In this way, the system learns the movements during camera work and can imitate the camera work by a cinematographer depending on the present shooting scene.

According to a further embodiment of the method according to the invention, it may be provided that determining the control parameters includes determining camera parameters which are used to control the camera. In this way, the aperture setting and the shutter speed setting can also be automated. This enables scenic filming in an advantageous way. In this way, for example, higher frame rates can be set for dynamic shooting scenes. The camera's focusing unit can also be controlled in this way. This makes it possible, for example, to automatically set the zoom parameters for shooting a romantic scene. Overall, this enables a significantly increased degree of automation of the shooting process.

According to a further embodiment of the method according to the invention, it may be provided that camera parameters are determined by a method based on machine learning, wherein a system previously trained with training data is used which was trained during a training process with image data or video sequences as well as camera parameters. Furthermore, the initially described object of the invention is achieved by proposing a camera robot for shooting a video sequence, wherein the camera robot comprises:
a chassis that can be moved on a surface;
a camera for shooting the video sequence;
a holding device for connecting the camera to the chassis as well as for orienting the camera relative to the chassis; and
a control unit configured to control the chassis, the holding device and/or the camera, wherein the control unit is configured to determine the control parameters of the control unit depending on a currently available characteristic shooting scene.

The chassis of the camera robot can be similar to known vacuum cleaner robots. The chassis may have three wheels, two of which are mechanically driven. Alternatively, the chassis can also have four or more wheels. The camera can have a zoom lens which can be controlled by the control unit. The holding device can in particular have a linear motor-like device for adjusting the vertical position of the camera. Furthermore, the holding device can have one or two pan motors which serve to align the pan angle of the camera. The control unit can be designed as a microcontroller which is used to set all relevant control parameters.

According to an exemplary embodiment of the camera robot according to the invention, it may be provided that the holding device is configured to adjust the vertical position of the camera and/or the pan angle of the camera. In this way, it is possible to adjust the camera particularly flexibly and thus to use all the degrees of freedom that are also available to a cinematographer with classic shooting methods.

According to a further embodiment of the camera robot according to the invention, it may be provided that the camera robot comprises at least one LIDAR sensor configured to detect objects in the environment of the camera robot.

Furthermore, the camera robot can comprise one or more IMU sensors (Inertial Measurement Unit) configured to detect the position, speed, acceleration and/or orientation of the camera.

It may also be provided that the camera robot comprises one or more LIDAR sensors that are set up to detect a room and objects located therein. In particular, LIDAR sensors can be used to detect any obstacles in the space so that (if the previously calculated ideal route leads to a collision with the obstacle) an alternative route can be provided for the camera robot.

In addition, the camera robot can have ultrasonic sensors and/or radar sensors configured to detect a space and any objects located therein.

According to an embodiment of the camera robot according to the invention, it may be provided that additional image sensors are provided which are used to analyze a room and detect obstacles in the room. For this purpose, image shots can be analysed with the aid of an object recognition algorithm so that typical obstacles (cables, table edge, etc.) can be detected and such obstacles can be taken into account when calculating the route for the camera travel.

### BRIEF DESCRIPTION OF THE DRAWINGS

In the following, the present invention is described with reference to the Figures. The Figures show the following:

FIG. 1 shows an embodiment of the method according to the invention,

FIG. 2 shows an operating unit for selecting the desired operating mode,

FIG. 3 shows the operating unit shown in FIG. 2 when selecting the shooting scene,

FIG. 4 shows an illustration of a database with predefined shooting scenes and control parameter sets associated with the shooting scenes,

FIG. 5 shows a first exemplary embodiment of the camera robot according to the invention,

FIG. 6 shows a second exemplary embodiment of the camera robot according to the invention, and

FIGS. 7(a), 7(b), 7(c), and 7(d) show different camera travels.

### DESCRIPTION OF THE INVENTION

FIG. 1 shows a first exemplary embodiment of method 100 according to the invention for controlling a camera robot. In a first step 110, a characteristic shooting scene is determined. As will be explained in more detail in connection with the following figures, the characteristic shooting scene 110 can be determined in particular by evaluating a user input or using a method based on machine learning. After the characteristic shooting scene has been determined, in a second step 120 of method 100 according to the invention, control parameters of the control unit are determined depending on the determined characteristic shooting scene. By using the determined control parameters, the camera robot is provided with all control parameters required for shooting a video sequence taking into account the determined shooting scene. The control parameters can in particular include parameters that serve to control the chassis, the holding device and/or the camera.

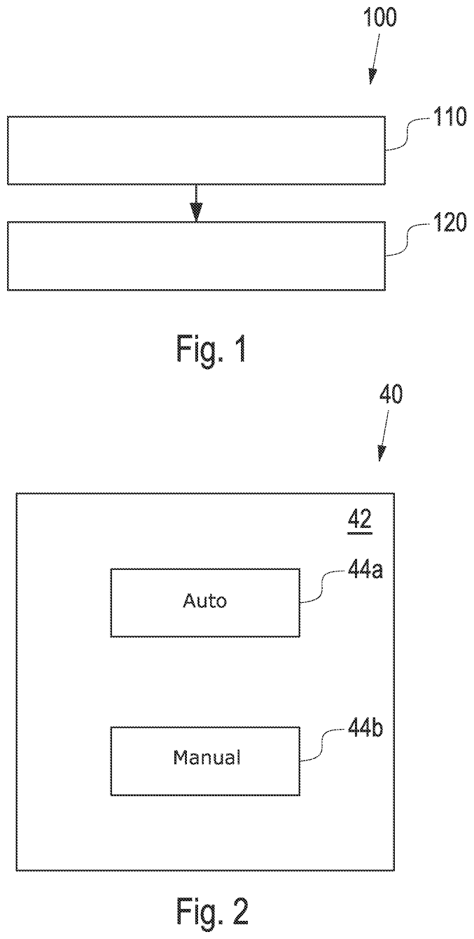

FIG. 2 shows an operating unit 40 allowing the user to select the desired operating mode. Operating unit 40 can be configured as a part of the camera robot and can comprise a touchscreen with a display panel 42. As an alternative, operating unit 40 can be configured as a separate unit, for example in the form of a tablet or a smartphone. As illustrated in FIG. 2, display panel 42 shows a first selection button 44a for selecting a first (automatic) operating mode and a second selection button 44b for selecting a second (manual) operating mode. According to the illustrated exemplary embodiment, the user can specify by input whether he wants an automatic or a manual operating mode.

In the automatic operating mode, the shooting scene is performed by using a method based on machine learning. The user does not have to specify which shooting scene is currently available. As an alternative, the user can opt for the manual operating mode according to the exemplary embodiment illustrated in FIG. 2. In the manual operating mode, the user can actively select the desired shooting scene. This allows the user to actively intervene in the shooting process and thus ensure that the shooting scene the user wants is taken into account when controlling or driving the camera robot.

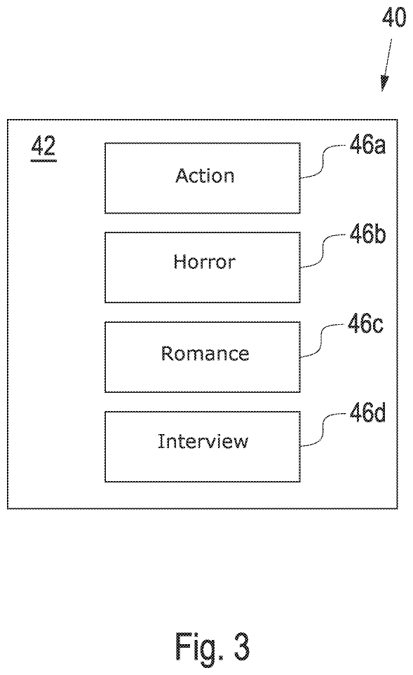

FIG. 3 shows a further user interface that is displayed to the user if he has previously chosen the manual operating mode. As illustrated in FIG. 3, a first selection button 46a, a second selection button 46b, a third selection button 46c and a fourth selection button 46d are displayed in display panel 42 of operating unit 40 for manually selecting the desired shooting scene. Thus, the user can select from four different shooting scenes in the exemplary embodiment illustrated here. It is taken for granted here that the number of shootings scenes available to the user can be varied as desired within the scope of the present invention. It may also be provided that the shooting scenes are subdivided into sub-shooting scenes in order to be able to distinguish between different scenes of a category. In this way, the subtleties of a shot are taken into account in the context of a specific shooting scene. For example, according to alternative embodiments of the invention, it may be provided that the user first selects a “dance” shooting scene and is then given the option to select a specific type of dance (e.g. hip-hop, tango or flamenco).

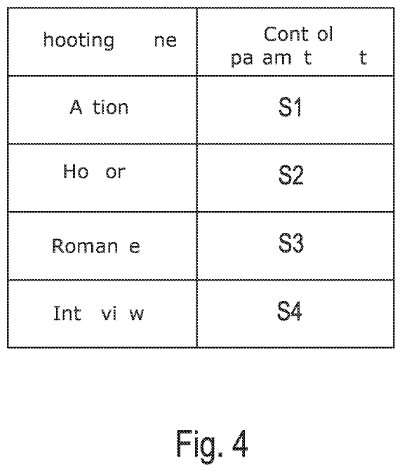

FIG. 4 exemplarily shows a database in which four different shooting scenes and the control parameter sets associated with the shooting scenes are illustrated. Regardless of whether the corresponding shooting scene has been recognized automatically or selected manually, the control parameters necessary for shooting a video sequence can be loaded from the database shown. If, for example, a romantic shooting scene has been determined, the control parameter set S3 is loaded from the database and transmitted to the control unit. The control unit is thus able to control the chassis, the holding device and/or the camera. For example, the control parameter set can include data that code a camera travel typical of the romantic shooting scene. For example, the control parameter set can include the location data that causes the camera robot to travel 360° around two actors. In this context, the control parameter set can, for example, exclusively comprise data for controlling the chassis or, alternatively, comprise both chassis control data and holding device control data. In addition, the control parameter set can also include control data for controlling the camera.

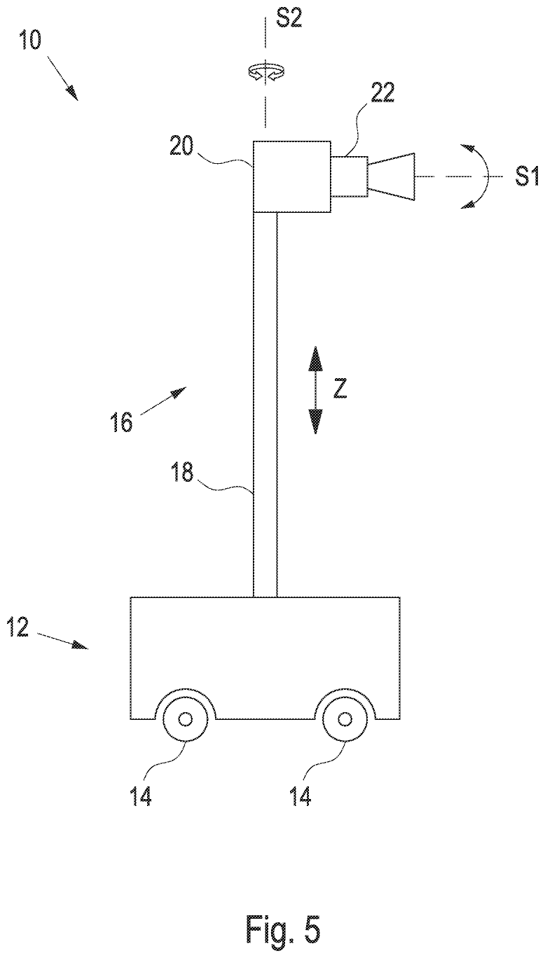

FIG. 5 shows a first exemplary embodiment of camera robot 10 according to the invention. Camera robot 10 comprises a chassis 12 having four controllable wheels 14, for example. In addition, camera robot 10 comprises a holding device 16. In the exemplary embodiment shown in FIG. 5, holding device 16 comprises a holding rod 18 connected to chassis 12 as well as a holding element 20 connected to holding rod 18. According to the illustrated exemplary embodiment, a camera 22 used for shooting the video sequence is connected to holding element 20. Here, camera 22 is configured so that it can be shifted vertically (along the z-axis). In addition, camera 22 is adapted to rotate about a first axis S1 and to pan about a second axis S2. The height adjustment of camera 22 can be carried out via a linear motor-like device, for example. It may be provided that the holding rod 18 is designed as an electric lifting column configured to position camera 22 along the z-axis, for example. Two electric rotary motors can be used for the rotation or panning of camera 22, which enable the rotation and pan movement the camera 22 about the axes S1 and S2. In this way, camera 22 can be shifted in height as well as panned and, in addition, can also be moved on a surface. This allows camera 22 to be positioned and oriented as desired. Chassis 12 can also have an electric drive configured to control all or only a selection of the wheels 14. In doing so, chassis 12 can be moved forwards or backwards. A rotation of chassis 12 can also be achieved by moving two opposing wheels 14 asynchronously. According to an embodiment, it may be provided that chassis 12 has a total of three wheels 14, of which two wheels are designed as driven rear wheels and the third wheel is designed as a front wheel that cannot be driven. In this case, the front wheel is adapted to be rotatable. By moving one rear wheel in the forward direction and the other rear wheel in the reverse direction, chassis 12 can be made to rotate about a central axis. According to an embodiment, chassis 12 can be substantially designed as a robot vacuum cleaner.

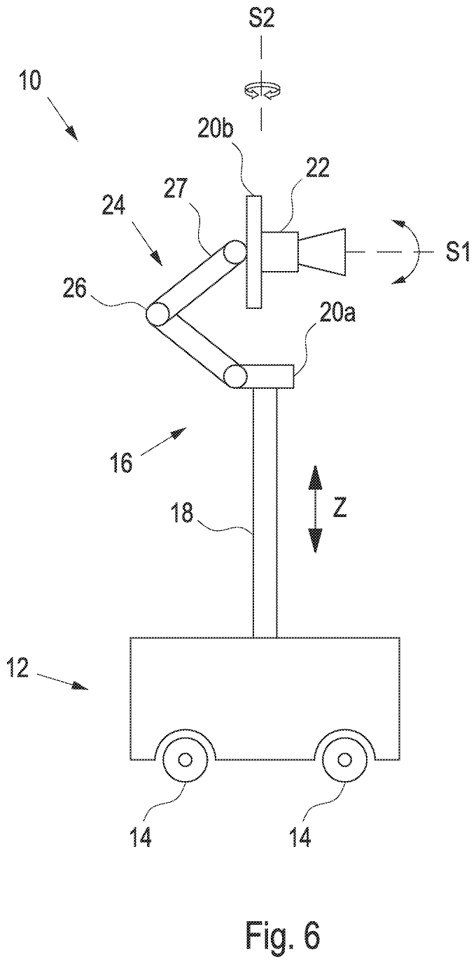

FIG. 6 shows a second exemplary embodiment of camera robot 10 according to the invention. Chassis 12 and the wheels 14 can be designed analogously to the exemplary embodiment shown in FIG. 5. In contrast, holding device 16 in the second exemplary embodiment is designed differently than in the exemplary embodiment shown in FIG. 5. Holding device 16 comprises a holding rod 18, a first holding element 20a connected to the holding rod 18, a jointed arm 24 connected to the first holding element 20a and a second holding element 20b, and a camera 22 connected to the second holding element 20b. Jointed arm 24 comprises several joints 26 and several joint rods 27. Jointed arm 24 allows camera 22 to be moved in its height (along the z-axis). Moreover, jointed arm 24 allows camera 22 to be pan or rotate about the first axis S1 and the second axis S2. This makes the camera particularly flexible to adjust. Furthermore, jointed arm 24 can serve to compensate for any vibrations that lead to undesired effects. The exemplary embodiment illustrated in FIG. 6 shows that camera robot 10 according to the invention enables flexible adjustment of the camera position as well as precise alignment of the camera, wherein camera 22 can be controlled as desired by the determined control parameters.

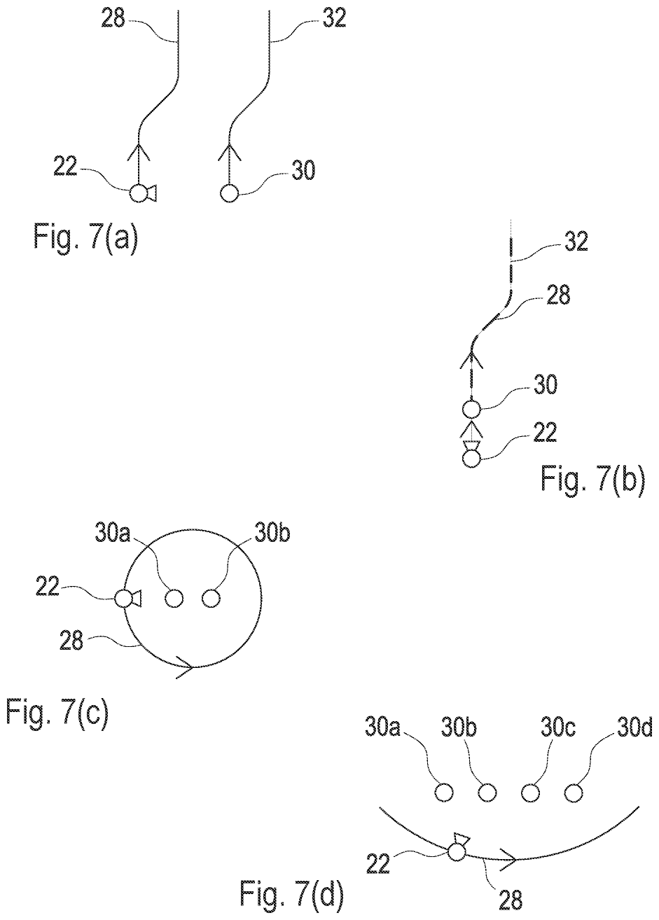

FIG. 7 shows various camera travels that can be carried out during shooting. FIG. 7(a) shows a parallel travel. As can be seen in this Figure, camera 22 is moved along a camera trajectory 28. In doing so, camera 22 is directed at a protagonist 30. Protagonist 30 may be an actor or a presenter, for example. Camera 22 can detect protagonist 30 and follow its movement along protagonist trajectory 32. In the shooting shown in FIG. 7(a), only control parameters to control the chassis are required. The orientation of camera 22 can remain unchanged in the example shown. The camera travel shown in FIG. 7(a) can be carried out, for example, when a presentation shooting scene has been determined.

FIG. 7(b) shows a tracking travel. As can be seen in this Figure, camera 22 follows protagonist 30. As protagonist 30 moves along protagonist trajectory 32, camera robot 10 is controlled such that camera 22 is moved along camera trajectory 28. The camera travel shown in this Figure can be carried out, for example, if an action shooting scene has been determined beforehand. During this travel, both the chassis and holding device 22 are controlled.

FIG. 7(c) shows a 360° travel of camera 22. Here, camera robot 10 is controlled such that camera 22 is moved along a circular camera trajectory 28, wherein camera trajectory 28 leads around a first protagonist 30a and a second protagonist 30b. At the same time, the pan angle of camera 22 is changed so that camera 22 is permanently directed at the protagonists 30a, 30b. To provide the camera travel shown in FIG. 7(c), one the one hand, control parameters for controlling the chassis and, on the other hand, control parameters for controlling the holding device are thus required. The camera travel shown in this Figure can be carried out, for example, if a romantic shooting scene has been determined beforehand.

FIG. 7(d) shows a further camera travel. As can be seen in this Figure, four different protagonists 30a, 30b, 30c, 30d are provided in this shooting scene, suggesting, for example, that this is a panel discussion. Camera robot 10 can recognize this shooting scene from the number, the posture, the facial expressions and the direction of gaze of the protagonists 30a, 30b, 30c, 30d, for example. Thus, once a panel discussion shooting scene has been recognized, camera 22 can be moved fully automatically along camera trajectory 28. In this example it is also provided that control parameters for controlling the chassis as well as control parameters for controlling the holding device are provided.

### LIST OF REFERENCE NUMERALS

10 camera robot
12 chassis
14 wheel
16 holding device
18 holding rod
20 holding element
20a first holding element
20b second holding element
22 camera
24 jointed arm
26 joint
27 joint rod
28 camera trajectory
30 protagonist
30a first protagonist
30b second protagonist
30c third protagonist
30d fourth protagonist
32 protagonist trajectory
40 operating unit
42 display panel
44a first selection button for selecting a first operating mode
44b second selection button for selecting a second operating mode
46a first selection button for manually selecting a first shooting scene
46b second selection button for manually selecting a second shooting scene
46c third selection button for manually selecting a third shooting scene
46d fourth selection button for manually selecting a fourth shooting scene
100 method for controlling a camera robot
110 first step of method according to the invention
120 second step of method according to the invention
S1 first axis
S2 second axis

1. A method for controlling a camera robot for shooting a video sequence, the camera robot comprising:
a chassis that can be moved on a surface;
a camera for shooting the video sequence;
a holding device; for connecting the camera to the chassis as well as for orienting the camera relative to the chassis; and
a control unit configured to control the chassis, the holding device and/or the camera;
the method; comprising the following steps:
determining a characteristic shooting scene; and
determining control parameters of the control unit depending on the determined characteristic shooting scene.

2. The method according to claim 1, wherein the characteristic shooting scene is determined by evaluating a user input.

3. The method according to claim 1, wherein the characteristic shooting scene is determined by a method based on machine learning.

4. The method according to claim 1, wherein, when determining the characteristic shooting scene, a system previously trained with training data is used which was trained during a training process with image data or video sequences and corresponding labels which identify the affiliation of the image data or video sequences to the shooting scenes.

5. The method according to claim 1, wherein the characteristic shooting scene comprises one of the following shooting scenes:
action,
horror,
romance,
dance,
presentation,
interview, or
panel discussion.

6. The method according to claim 1, wherein the control parameters are determined depending on the determined characteristic shooting scene by a method based on machine learning, wherein in particular a system previously trained with training data is used which was trained during a training process with image data or video sequences as well as shooting scene information and control parameters.

7. The method according to claim 1, wherein determining the control parameters comprises determining chassis control parameters which are used to control the movement of the chassis on the surface along a determined route.

8. The method according to claim 1, wherein chassis control parameters are determined by a method based on machine learning, wherein a system previously trained with training data is used which was trained during a training process with image data or video sequences as well as chassis control parameters.

9. The method according to claim 1, wherein objects located in the environment of the camera robot are detected, and wherein chassis control parameters are determined depending on the detected objects and/or their position.

10. The method according to claim 1, wherein the objects located in the environment of the camera robot are detected by using a LIDAR sensor.

11. The method according to claim 1, wherein the holding device is configured to adjust the position of the camera along a vertical axis and/or a tilt angle of the camera, and that determining the control parameters comprises determining holding device control parameters used to control the position of the camera along a vertical axis and/or the tilt angle of the camera.

12. The method according to claim 1, wherein the holding device control parameters are determined by a method based on machine learning, wherein a system previously trained with training data is used which was trained during a training process with image data or video sequences as well as holding device control parameters.

13. A camera robot for shooting a video sequence, comprising
a chassis that can be moved on a surface;
a camera for shooting the video sequence;
a holding device for connecting the camera to the chassis as well as for orienting the camera relative to the chassis; and
a control unit configured to control the chassis, the holding device and/or the camera, wherein the control unit is configured to determine the control parameters of the control unit depending on a currently available characteristic shooting scene.

14. The camera robot according to claim 13, wherein the holding device is configured to adjust the vertical position of the camera and/or the tilt angle of the camera.

15. The camera robot according to claim 13, wherein at least one LIDAR sensor configured to detect objects located in the environment of the camera robot.
